(19) United States
(12) Patent Application Publication
(10) Pub. No.: US 2021/0004695 A1
(43) Pub. Date: Jan. 7, 2021

(54) ELECTRONIC APPARATUS AND INFORMATION PROCESSING SYSTEM

(71) Applicant: KABUSHIKI KAISHA TOSHIBA, Minato-ku (JP)

(72) Inventors: Tsukasa IKE, Shinagawa (JP); Kazunori IMOTO, Kawasaki (JP); Yasunobu YAMAUCHI, Yokohama (JP)

(73) Assignee: KABUSHIKI KAISHA TOSHIBA, Minato-ku (JP)

(21) Appl. No.: 16/809,775

(22) Filed: Mar. 5, 2020

(30) Foreign Application Priority Data
Jul. 3, 2019 (JP) 2019-124455

(51) Int. Cl. G06N 5/04; G06F 1/16; G16H 40/67

(52) U.S. Cl. CPC G06N 5/04; G06N 20/00; G16H 40/67; G06F 1/163

(57) ABSTRACT

According to one embodiment, an electronic apparatus is wearable or portable by a user, and includes one or more sensors, one or more processors, and a transmitter. The one or more processors acquires one or more pieces of first time-series sensor data, using the one or more sensors. The one or more processors detects a candidate for at least one of a behavior or a state of the user, using at least one of the one or more pieces of first time-series sensor data. The transmitter transmits, when the candidate is detected, a data subset of a first period, of at least one of the one or more pieces of first time-series sensor data, to an external processing device, in accordance with the candidate.

### CROSS-REFERENCE TO RELATED APPLICATIONS

This application is based upon and claims the benefit of priority from Japanese Patent Application No. 2019-124455, filed Jul. 3, 2019, the entire contents of which are incorporated herein by reference.

### FIELD

Embodiments described herein relate generally to an electronic apparatus and an information processing system for recognizing a user's behavior and state.

### BACKGROUND

In recent years, wearable devices such as activity trackers and smartwatches have been prevalent. Sensors in the wearable devices measure the acceleration, temperatures and humidity, physiological signals, etc., of users. Techniques for recognizing the behaviors and states of the users, using signals measured by these sensors, also have been actively developed.

The wearable devices are used, not only by general consumers, but also by operators engaged in various operations such as manufacturing, logistics, and field maintenance. Especially when they are used in the industrial fields, it is often required that the sizes of the wearable devices be smaller so as not to hinder operations. The computational performance and the battery capacity of these wearable devices may be low.

### BRIEF DESCRIPTION OF THE DRAWINGS

FIG. 1 is a block diagram illustrating an example of a configuration of an information processing system comprising an electronic apparatus according to a first embodiment and an external processing device.

FIG. 2 is a block diagram illustrating an example of a system configuration of the electronic apparatus of the first embodiment.

FIG. 3 is a block diagram illustrating an example of a functional configuration of the electronic apparatus of the first embodiment and the external processing device.

FIG. 4 is a diagram illustrating an example of a processing sequence performed by the electronic apparatus of the first embodiment and the external processing device.

FIG. 5 is a diagram for explaining data acquired by the electronic apparatus of the first embodiment and the external processing device.

FIG. 6 is a diagram illustrating an example in which a candidate indicating that a user may be in a specific state (for example, a heatstroke state) is detected by the electronic apparatus of the first embodiment.

FIG. 7 is a diagram illustrating an example of a data subset generated in accordance with the candidate of FIG. 6 by the electronic apparatus of the first embodiment.

FIG. 8A is a diagram illustrating an example of a screen image displayed by the electronic apparatus of the first embodiment or the external processing device.

FIG. 8B is a diagram illustrating an example of another screen image displayed by the electronic apparatus of the first embodiment or the external processing device.

FIG. 9 is a block diagram illustrating an example of a functional configuration of an electronic apparatus according to a second embodiment and an external processing device.

FIG. 10 is a diagram illustrating an example of a processing sequence performed by the electronic apparatus of the second embodiment and the external processing device.

FIG. 11 is a diagram for explaining levels of heat stress indices used as the degrees of urgency by the electronic apparatus of the second embodiment.

FIG. 12A is a diagram illustrating an example of a screen image displayed by the electronic apparatus of the second embodiment or the external processing device.

FIG. 12B is a diagram illustrating an example of another screen image displayed by the electronic apparatus of the second embodiment or the external processing device.

FIG. 12C is a diagram illustrating an example of still another screen image displayed by the electronic apparatus of the second embodiment or the external processing device.

FIG. 13 is a block diagram illustrating an example of a functional configuration of an electronic apparatus according to a third embodiment and an external processing device.

FIG. 14 is a diagram illustrating an example of a processing sequence performed by the electronic apparatus of the third embodiment and the external processing device.

FIG. 15 is a diagram illustrating an example of a screen image displayed by the electronic apparatus of the third embodiment.

FIG. 16 is a block diagram illustrating an example of a configuration of an information processing system comprising an electronic apparatus according to a fourth embodiment and an external processing device.

FIG. 17 is a block diagram illustrating an example of a functional configuration of the electronic apparatus of the fourth embodiment, another electronic apparatus, and the external processing device.

FIG. 18 is a diagram illustrating an example of a processing sequence performed by the electronic apparatus of the fourth embodiment, the other electronic apparatus, and the external processing device.

### DETAILED DESCRIPTION

Embodiments will be described hereinafter with reference to the accompanying drawings. In the drawings, the same elements are denoted by the same reference symbols, and a duplicate explanation is omitted.

In general, according to one embodiment, an electronic apparatus is wearable or portable by a user, and includes one or more sensors, one or more processors, and a transmitter. The one or more processors acquires one or more pieces of first time-series sensor data, using the one or more sensors. The one or more processors detects a candidate for at least one of a behavior or a state of the user, using at least one of the one or more pieces of first time-series sensor data. The transmitter transmits, when the candidate is detected, a data subset of a first period, of at least one of the one or more pieces of first time-series sensor data, to an external processing device, in accordance with the candidate.

### First Embodiment

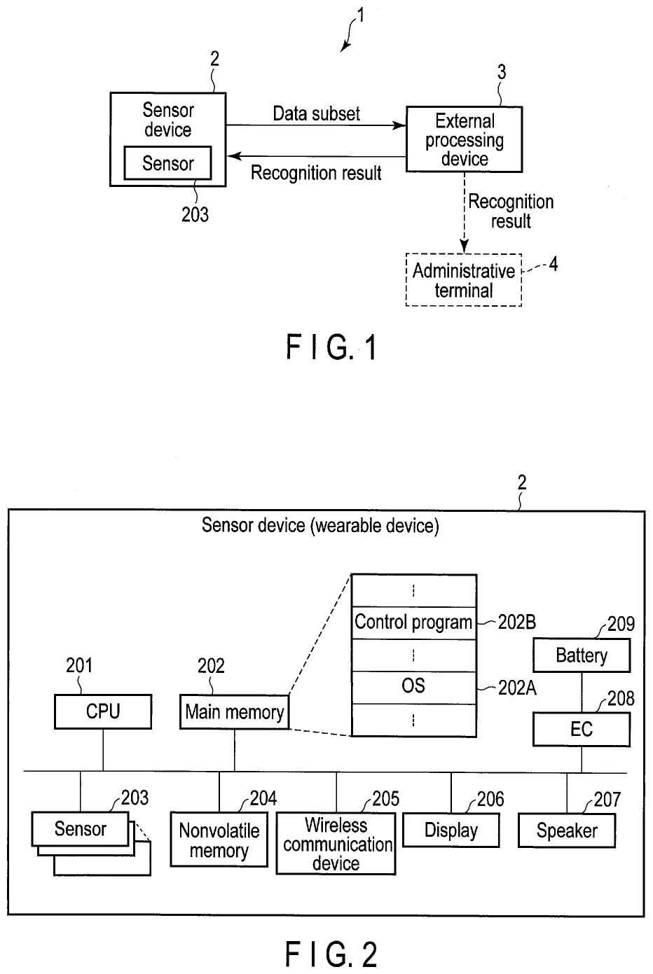

First, a configuration of an information processing system including an electronic apparatus according to a first embodiment will be described with reference to FIG. 1. The information processing system 1 is a system for recognizing a behavior and a state of a recognition target user, and is also referred to as a behavior/state recognition system. The user's behavior and state to be recognized include various behaviors and states indicating bad condition and the occurrence or a symptom of an accident, such as unsteadiness, a fall and a heatstroke state. The information processing system 1 may include an electronic apparatus 2 including one or more sensors 203, and an external processing device 3. Hereinafter, the electronic apparatus 2 is also referred to as a sensor device 2.

The sensor device 2 may be realized as a wearable device which can be worn on the wrist, the ankle, the neck, the waist, the head, etc., of the recognition target user or a portable device which can be carried by the recognition target user (for example, a smartphone). Each of the one or more sensors 203 in the sensor device 2 generates various signals (or data) related to the behavior and state of the user wearing or carrying the sensor device 2. The one or more sensors 203 are sensors for measuring, for example, acceleration, angular velocity, geomagnetism, air pressure, temperature, humidity, and physiological signals (myoelectric potential, heartbeat, pulse wave, etc.). The sensor device 2 can acquire time-series sensor data based on the generated signals.

In the following description, for the sake of clarification, a case where the sensor device 2 is a wearable device worn by the recognition target user will be mainly described as an example. In addition, the recognition target user will also be simply referred to as a user.

The external processing device 3 is an information processing apparatus, and may be realized as a server computer, etc. The external processing device 3 and the sensor device 2 can mutually transmit and receive data via a network. The sensor device 2 may transmit and receive data to and from the external processing device 3 through, for example, wireless connection using a wireless LAN or Bluetooth (registered trademark).

The sensor device 2 is used, not only by a general consumer, but also by an operator engaged in various operations such as manufacturing, logistics, and field maintenance. When the sensor device 2 is worn by the general consumer, time-series sensor data acquired with the one or more sensors 203 indicates the number of steps, exercise intensity, a heart rate, etc., and is used for healthcare, etc. In contrast, when the sensor device 2 is worn by the operator, time-series sensor data acquired with the one or more sensors 203 is used to classify operations in a workplace to make an analysis for improving productivity, used to secure the safety of the operator through detection of a fall, estimation of the risk of heatstroke, etc., and used for other purposes. In this manner, the time-series sensor data can also be used in the industrial fields.

Especially when the sensor device 2 is used in the industrial fields, it can be required that the size of the sensor device 2 be smaller so that the wearing of the sensor device 2 will not hinder the user's operation. Thus, restrictions may be imposed on the size of each component, such as a processing unit and a battery in the sensor device 2. To drive the sensor device 2 for a long time in this case, it is necessary to reduce power consumed by, for example, the processing unit (i.e., an SoC, a processor, etc.). However, this means that the processing device can exhibit only low performance. Accordingly, there may be great restrictions on, for example, the sensor device 2's computational performance (or resources) for a process for recognizing the user's behavior and state.

In the present embodiment, the process for recognizing the user's behavior and state is executed by the external processing device 3. That is, a high-level recognition process having great computational amount is executed by the external processing device 3, which is free of the above restrictions imposed to reduce power consumption.

When the external processing device 3 executes the process for recognizing a behavior/state, the sensor device 2 needs to transmit information used for the recognition, such as signals, etc., measured by the one or more sensors 203, to the external processing device 3. If the sensor device 2 and the external processing device 3 are connected by wire, the user needs to carry not only the sensor device 2 but also the external processing device 3, which hinders the operation. It is therefore preferable that the sensor device 2 can transmit the information to the external processing device 3 by wireless communication so as not to hinder the operation.

Power required for wireless communication increases when the amount of transmitted data increases. Thus, in order to drive the sensor device 2 for a long time, it is preferable that the amount of data to be transmitted be reduced.

One of the methods for reducing the amount of data to be transmitted is, for example, a method of maintaining only frequency components necessary for the detection of an event to be recognized (for example, detection of a pulse from a physiological signal) of sensor data (signal) and removing the other frequency components, thereby compressing the sensor data. By transmitting the compressed sensor data to the external processing device 3, power required for wireless communication can be reduced in the sensor device 2.

However, when the user's behavior/state is recognized, multiple types of behavior/state may be recognized in parallel from sensor data of an characteristic frequency components (that is, frequency components necessary for recognition) vary according to the behaviors/states to be recognized in parallel. For example, when several behaviors of the user are to be recognized, low frequency components exhibit a characteristic pattern in the case of repeated actions such as walking, whereas high frequency components exhibit a characteristic pattern in the case of actions involving a rapid speed change of the body (for example, arms and legs), such as starting or finishing an action or touching an object. Thus, it is hard to select necessary frequency components without lowering the accuracy in recognizing the behaviors/states to be recognized in parallel. Accordingly, it is hard to apply the method of compressing sensor data by thinning out frequency components to the present embodiment, which is intended for the recognition of the user's behavior/state.

Therefore, in the present embodiment, the sensor device 2 detects a candidate for at least one of a behavior and a state (hereinafter, also referred to as a behavior/state candidate) from one or more pieces of time-series sensor data, and when the candidate is detected, transmits a data subset of a first period of at least one of the one or more pieces of time-series sensor data to the external processing device 3, in accordance with the candidate. The sensor device 2 transmits, not all the time-series sensor data, but the data subset necessary for the recognition of a behavior/state performed by the external processing device 3, and thus, power required for communication can be reduced. Moreover, the external processing device 3 has higher computing capability than that of the sensor device 2, and thus can accurately recognize at least one of a behavior and a state (hereinafter, also referred to as a behavior/state), using the data subset. The sensor device 2 thereby can be driven for a long time and can acquire a highly accurate recognition result of a behavior/state.

More specifically, the sensor device 2 acquires one or more pieces of time-series sensor data with the one or more sensors 203, for example, in real time. The sensor device 2 detects a candidate for the user's behavior/state to be recognized, using at least one of the acquired one or more pieces of time-series sensor data. Then, the sensor device 2 transmits a data subset of a first period of the at least one of the one or more pieces of time-series sensor data to the external processing device 3, in accordance with the detected candidate. The sensor device 2 selects the at least one piece of time-series sensor data of the one or more pieces of time-series sensor data, on the basis of the detected candidate, and acquires at least part of each of the selected at least one piece of time-series sensor data as the data subset. That is, the acquired data subset is a subset of each of the selected at least one piece of time-series sensor data.

The external processing device 3 receives the data subset, and uses the data subset to recognize the user's behavior/state.

FIG. 2 illustrates an example of a system configuration of the sensor device 2. The sensor device 2 includes a CPU 201, a main memory 202, the one or more sensors 203, a nonvolatile memory 204, a wireless communication device 205, an embedded controller (EC) 208, etc.

The CPU 201 is a processor that controls the operation of various components in the sensor device 2. The CPU 201 executes various programs loaded from the nonvolatile memory 204, which is a storage device, into the main memory 202. These programs include an operating system (OS) 202A and various application programs. The application programs include a control program 202B for processing time-series sensor data acquired by each of the one or more sensors 203. The control program 202B includes instructions for acquiring time-series sensor data with each of the one or more sensors 203, detecting a candidate for the user's behavior/state using the time-series sensor data, and transmitting a data subset of the time-series sensor data to the external processing device 3, in accordance with the detected candidate.

The wireless communication device 205 is a device configured to perform wireless communication. The wireless communication device 205 includes a transmitter that transmits a signal wirelessly and a receiver that receives a signal wirelessly. The wireless communication device 205A may adopt any wireless communication method such as a wireless LAN, Bluetooth, etc.

The EC 208 is a single-chip microcomputer including an embedded controller for power management. The EC 208 controls power supplied from a battery 209 to each part in the sensor device 2.

The sensor device 2 may further include a display 206 and a speaker 207. In this case, the CPU 201 controls the display 206 and the speaker 207. A display signal generated by the CPU 201 is transmitted to the display 206. The display 206 displays a screen image based on the display signal. Similarly, a sound signal generated by the CPU 201 is transmitted to the speaker 207. The speaker 207 outputs a sound based on the sound signal.

Alternatively, the sensor device 2 may be connected to another electronic apparatus that can output video and sounds (for example, a head-mounted display) wirelessly or by wire. In this case, the other electronic apparatus can be used to display a screen image and output sounds.

While the external processing device 3 may have the same system configuration as that of the sensor device 2, the performance of at least part of the configuration (CPU, main memory, etc.) in the external processing device 3 may be higher than that of the corresponding structure in the sensor device 2. Moreover, the external processing device 3 may include a wired communication device in addition to or instead of a wireless communication device.

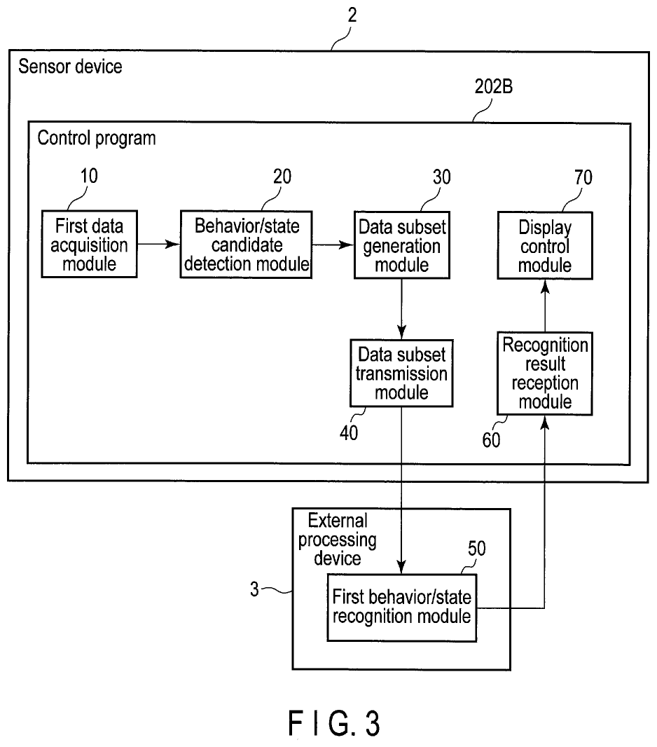

FIG. 3 illustrates an example of a functional configuration of the sensor device 2 and the external processing device 3. The control program 202B executed by the sensor device 2 includes a first data acquisition module 10, a behavior/state candidate detection module 20, a data subset generation module 30, a data subset transmission module 40, a recognition result reception module 60, and a display control module 70. In addition, the external processing device 3 includes a first behavior/state recognition module 50. The first behavior/state recognition module 50 may be realized as a function of a program executed by the external processing device 3.

The first data acquisition module 10 acquires sensor data, which is necessary to recognize a behavior/state of the recognition target user, from the one or more sensors 203. The acquired sensor data is, for example, acceleration data, angular velocity data, geomagnetic data, air pressure data, temperature and humidity data, myoelectric potential data, pulse wave data, etc.

The first data acquisition module 10 may acquire multiple types of sensor data from sensors 203, respectively. Alternatively, multiple types (for example, multiple channels) of sensor data may be acquired from one sensor 203. For example, the first data acquisition module 10 may acquire sensor data of six channels including acceleration data of three channels corresponding to the direction components of acceleration and angular velocity data of three channels corresponding to the direction components of angular velocity, in parallel, from the one or more sensors 203. In addition, the acquired sensor data may be any type of sensor data that includes information effective in recognizing the user's behavior/state. Moreover, the one or more sensors 203 for acquiring sensor data may be sensors (devices) having any structure that can acquire information effective in recognizing the user's behavior/state.

For example, when one or more types (or channels) of sensor data are successively acquired, the first data acquisition module 10 generates time-series sensor data into which pieces of each type of sensor data are combined in a time series. One or more types of time-series sensor data are thereby acquired.

The behavior/state candidate detection module 20 detects a candidate for the user's behavior/state to be recognized, using generated one or more pieces of time-series sensor data. The behavior/state candidate detection module 20 detects a section in which a behavior/state to be recognized may occur, as a behavior/state candidate, on the basis of a pattern of at least one piece of time-series sensor data. Regarding a detected behavior/state candidate and a recognized behavior/state, the recognized behavior/state is more likely to actually occur than the behavior/state candidate.

The process for detecting a candidate for a behavior/state of a user can be executed with less power consumption than that of the process for recognizing the behavior/state. For example, when a candidate for a state in which the risk of a fall is high (for example, an unsteady state) is detected, the sensor device 2 including an acceleration sensor is worn on the user's waist. On the basis of time-series acceleration data acquired by the acceleration sensor, an inclination of the user's waist with respect to the direction of gravity is calculated, and on the basis of its variance value, it is determined whether there is a possibility of the unsteady state. That is, the behavior/state candidate detection module 20 detects the candidate for the unsteady state, for example, when the variance value of the waist's inclination is greater than or equal to a threshold value.

While the means of using a process having low computational amount has been herein described as an example of the means of detecting a behavior/state candidate with less power consumption, other means that can reduce power consumption may be used. For example, a processing unit that can execute a specific operation with less power consumption may be provided in the sensor device 2, and used to reduce the power consumption required for the detection of a behavior/state candidate.

When the behavior/state candidate detection module 20 detects a behavior/state candidate, the data subset generation module 30 and the data subset transmission module 40 transmit a data subset of a specific period, of at least one of one or more pieces of time-series sensor data, to the external processing device 3, in accordance with the behavior/state candidate.

More specifically, when a behavior/state candidate is detected, the data subset generation module 30 extracts a specific section from time-series sensor data, in accordance with the type of behavior/state candidate and the time (for example, time and date) when the behavior/state candidate is detected, and thereby generates a data subset for recognizing a behavior/state. For example, the data subset generation module 30 selects at least one piece of time-series sensor data from one or more pieces of time-series sensor data, in accordance with the type of behavior/state candidate. The data subset generation module 30 then acquires data of a specific period based on the time when the behavior/state candidate is detected, of the selected at least one piece of time-series sensor data, as the data subset. The selected at least one piece of time-series sensor data may include time-series sensor data used to detect the behavior/state candidate, or may include other time-series sensor data.

For example, a case where the action of detaching a screw cap is recognized will be described. In this case, the action of pulling the screw cap is first detected as a behavior/state candidate, using acceleration data. Then, the action of turning and unfastening the screw cap, prior to the point in time of the action of pulling the screw cap, is recognized, using angular velocity data. Through these detection and recognition, the action of detaching the screw cap is recognized. In this manner, when the action of detaching the screw cap is recognized, its behavior/state candidate is detected using acceleration data, and the action is recognized using angular velocity data other than the acceleration data. Thus, the data subset generation module 30 may acquire a data subset for recognizing a behavior/state from time-series sensor data other than time-series sensor data used to detect a behavior/state candidate.

The above specific period is a period having a specific length, for example, including the time when a behavior/state candidate is detected. In addition, this specific period may be a period not including the time when a behavior/state candidate is detected. For example, when the fall of the user is recognized, a shock at the time of a collision with the ground is detected as a candidate for the fall using acceleration data at a point in time, and the fall is recognized from a change in posture during the fall (that is, before the collision) using posture data of a period before the point in time. In this case, the data subset generation module 30 may acquire posture data of a period not including the time when the candidate for the fall is detected as a data subset.

The data subset transmission module 40 transmits the data subset generated by the data subset generation module 30 to the external processing device 3 via the wireless communication device 205.

The first behavior/state recognition module 50 of the external processing device 3 receives the data subset from the sensor device 2. The first behavior/state recognition module 50 recognizes a user's behavior/state to be recognized, using the data subset. The behavior/state to be recognized may be one type of behavior or state or may be a multiple types of behavior and state. The first behavior/state recognition module 50 may include multiple types of algorithm for recognizing the multiple types of behavior and state.

The first behavior/state recognition module 50 may store a recognition result, which includes information indicating at least one of a recognized behavior and state, in the external processing device 3 as a log of the user's behavior/state or may store the recognition result in, for example, a server in a cloud connected via a network.

In addition, the first behavior/state recognition module 50 may transmit a recognition result to the sensor device 2 so that, for example, the user can check the recognition result. In this case, the recognition result reception module 60 of the sensor device 2 receives the recognition result from the external processing device 3 via the wireless communication device 205. Then, the display control module 70 displays the recognition result on a screen of the display 206. Further, a sound for caution or warning based on the recognition result may be output from the speaker 207. Moreover, the display control module 70 may display information on a behavior/state candidate detected by the behavior/state candidate detection module 20 on the screen of the display 206, and a sound for caution or warning based on the information may be output from the speaker 207.

Alternatively, for example, when an operations supervisor manages the operation status and the condition of each operator, the first behavior/state recognition module 50 may transmit a recognition result to an administrative terminal 4 that is set near the operations supervisor or is carried by the operations supervisor. As in the case of the sensor device 2, the administrative terminal 4 may display the recognition result on its screen or may output a sound for caution or warning.

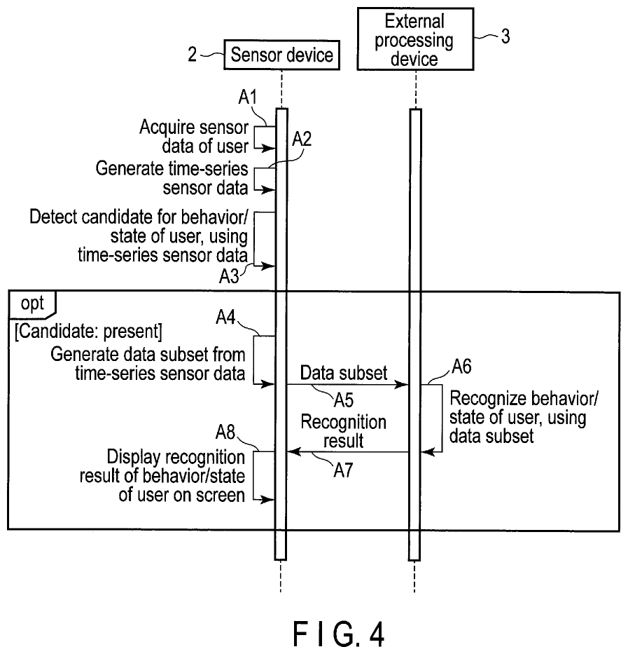

An example of a processing sequence performed by the sensor device 2 and the external processing device 3 will be described with reference to FIG. 4. As stated above, a case where the sensor device 2 is a wearable device worn by the recognition target user will be herein described as an example.

First, the first data acquisition module 10 of the sensor device 2 acquires one or more pieces of sensor data necessary to recognize the user's behavior/state, by using the one or more sensors 203 (A1). The first data acquisition module 10 combines the acquired one or more pieces of sensor data with pieces of sensor data acquired at past points in time, for each type (channel) of sensor data, and thereby generates one or more pieces of time-series sensor data (A2).

Then, the behavior/state candidate detection module 20 detects a behavior/state candidate, using at least one piece of time-series sensor data of the generated one or more pieces of time-series sensor data (A3). As described above, algorithms for detecting a behavior/state candidate are not necessarily of one type. For example, when there are multiple behaviors/states to be recognized or when there are multiple patterns of a behavior/state candidate corresponding to one behavior/state, the behavior/state candidates or the behavior/state candidate may be detected using multiple types of algorithm. When no behavior/state candidate is detected, the processing ends.

In contrast, when a behavior/state candidate is detected, the data subset generation module 30 extracts a specific section of time-series sensor data, using the type of detected behavior/state candidate and the time when the behavior/state candidate is detected, and thereby generates a data subset (A4). In this case, the type and the number of channels of time-series sensor data from which the data subset is extracted, and the length and the position of the extracted data subset may vary according to the detected behavior/state candidate. Then, the data subset transmission module 40 transmits the generated data subset to the external processing device 3 via the wireless communication device 205 (A5).

Next, the first behavior/state recognition module 50 of the external processing device 3 receives the data subset from the sensor device 2, and recognizes at least one of the user's behavior and state to be recognized, using the data subset (A6). Then, the first behavior/state recognition module 50 transmits the recognition result to the sensor device 2 (A7).

The recognition result reception module 60 of the sensor device 2 receives the recognition result from the external processing device 3 via the wireless communication device 205, and the display control module 70 displays the recognition result on the screen of the display 206 (A8). Further, a sound according to the recognition result may be output from the speaker 207. The recognition target user thereby can check the recognition result.

In addition, the recognition result may be stored in the external processing device 3 or another server, or may be transmitted to the administrative terminal 4 other than the sensor device 2, a portable information terminal (for example, a smartphone) carried by the user, etc. The recognition result is displayed on a screen of a display contained in or connected to any one of these devices (terminals), and an administrator such as an operations supervisor thereby can check the recognition result on each user.

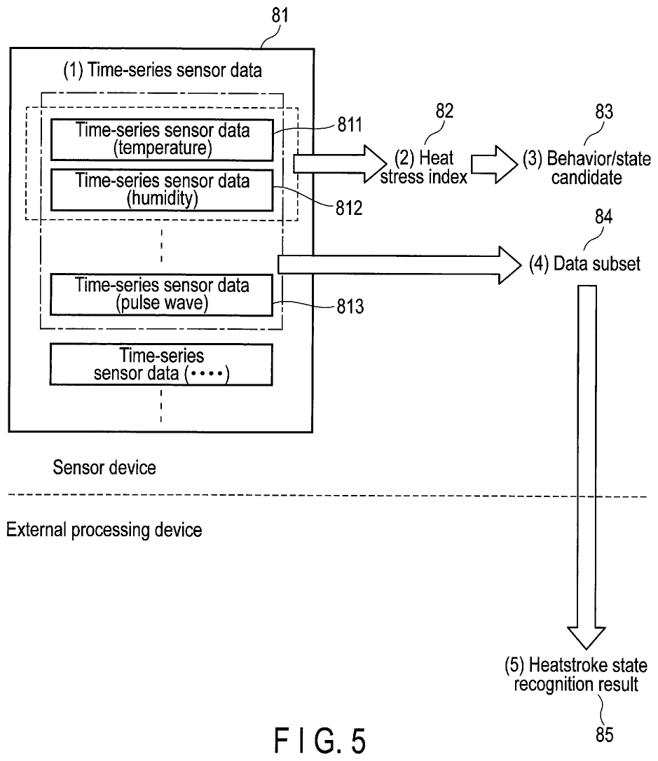

An example in which time-series sensor data is used to detect a behavior/state candidate and generate a data subset will be described with reference to FIG. 5 to FIG. 7. A case where the behavior/state to be recognized is a heatstroke state will be herein described as an example.

The magnitude of the risk of heatstroke may be broadly estimated by calculating a heat stress index (wet-bulb globe temperature [WBGT]) from the temperature and the humidity of a place. However, the magnitude of the actual risk varies according to the pulse rate or the quantity of body motion of the user, an environmental change in temperature or humidity, etc. Thus, a statistical process by machine learning (for example, deep learning), etc., using these pieces of information, is considered effective in estimating the risk accurately.

When the behavior/state to be recognized is a heatstroke state in the present embodiment, the sensor device 2 calculates a heat stress index, using time-series sensor data on temperature and humidity, and detects a candidate for the heatstroke state when the heat stress index exceeds a threshold value. In addition, The external processing device 3 performs a statistical process with machine learning, etc., further using the pulse rate, the quantity of body motion, etc., to recognize the heatstroke state (for example, the risk of heatstroke) accurately.

More specifically, as illustrated in FIG. 5, the first data acquisition module 10 of the sensor device 2 acquires one or more pieces of time-series sensor data 81 with the one or more sensors 203. The one or more pieces of time-series sensor data 81 may include multiple types of time-series sensor data, such as time-series sensor data 811 on temperature, time-series sensor data 812 on humidity, and time-series sensor data 813 on pulse wave. While an example in which time-series values constituting the time-series sensor data 811, 812, and 813 are measured at the same timing and at the same frequency (in this case, 20 times per second) is herein described, the timings and the frequencies of measurement may differ between the one or more sensors 203.

Next, the behavior/state candidate detection module 20 calculates a heat stress index 82, using the time-series sensor data 811 on temperature and the time-series sensor data 812 on humidity of the one or more pieces of time-series sensor data 81. When the calculated heat stress index 82 exceeds the threshold value, the behavior/state candidate detection module 20 detects the heat stress index 82 as a candidate for the heatstroke state, and acquires the time when data used to calculate the heat stress index 82 was measured.

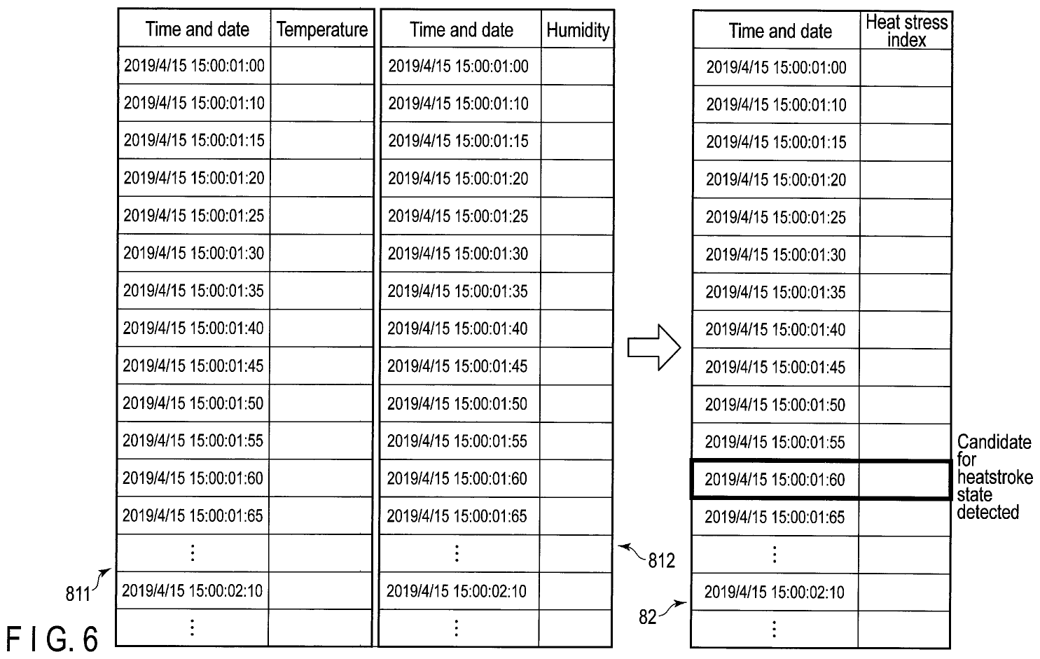

FIG. 6 illustrates an example in which the heat stress index 82 is calculated from the time-series sensor data 811 on temperature and the time-series sensor data 812 on humidity, and a candidate 83 for the heatstroke state is detected. The time-series sensor data 811 on temperature includes, for example, records each including a time and date and a temperature measured at the time and date in chronological order. In addition, the time-series sensor data 812 on humidity includes, for example, records each including a time and date and humidity measured at the time and date in chronological order.

The behavior/state candidate detection module 20 calculates the heat stress index 82, using a temperature and humidity at a time and date, and determines whether the heat stress index 82 exceeds the threshold value. Then, the behavior/state candidate detection module 20 detects the heat stress index 82 that exceeds the threshold value (in FIG. 6, a heat stress index at a time and date “2019/4/15 15:00:01:60”) as the candidate 83 for the heatstroke state.

On the basis of the fact that the type of detected candidate 83 is the heatstroke state, and a time and date corresponding to the candidate 83 (that is, a time and date when data used to detect the candidate 83 was measured), the data subset generation module 30 selects at least one piece of time-series sensor data from the one or more pieces of time-series sensor data 81, and generates a data subset of a specific period from the selected at least one piece of time-series sensor data.

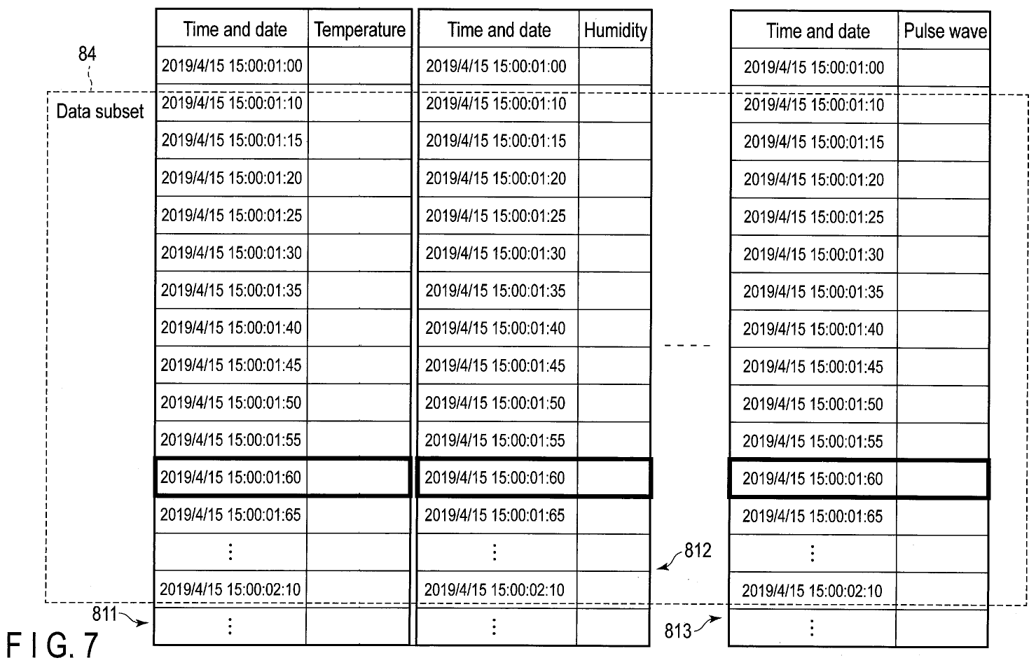

In an example illustrated in FIG. 7, the time-series sensor data 811 on temperature, the time-series sensor data 812 on humidity, and the time-series sensor data 813 on pulse wave for accurately recognizing the heatstroke state in the external processing device 3 are selected from the one or more pieces of time-series sensor data 81. In addition, a specific period including the time and date “2019/4/15 15:00:01:60” corresponding to the candidate 83 for the heatstroke state (for example, one second including the time and date “2019/4/15 15:00:01:60” in the middle) is extracted from each of the time-series sensor data 811, 812, and 813, and a data subset 84 is thereby generated.

The external processing device 3 recognizes the heatstroke state with high accuracy, using the generated data subset 84. An algorithm for the recognition is, for example, a regression algorithm based on machine learning. The external processing device 3 may estimate, for example, the magnitude of the risk of heatstroke with high accuracy. The external processing device 3 transmits the estimated magnitude of the risk of heatstroke to the sensor device 2, etc., as a recognition result.

It should be noted that FIG. 6 and FIG. 7 merely illustrate examples, and for example, time-series sensor data on acceleration may be further used to calculate the quantity of body motion or the time-series sensor data 813 on pulse wave may be further used to calculate the pulse rate in order to improve the accuracy in detecting the candidate 83 for the heatstroke state. Alternatively, in order to recognize the heatstroke state accurately by the external processing device 3, another type of time-series sensor data (for example, time-series sensor data on triaxial acceleration) may be further selected and used to generate the data subset 84.

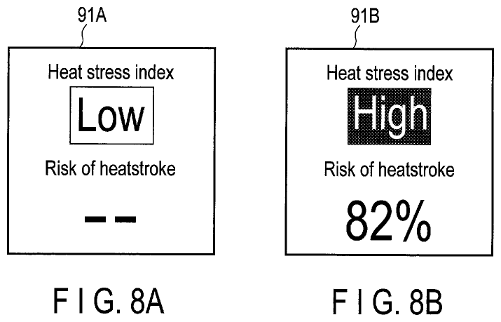

FIG. 8A and FIG. 8B illustrate an example of screen images showing a detection result of a behavior/state candidate and a recognition result of a behavior/state. The screen images illustrated in FIG. 8A and FIG. 8B may be displayed on, not only the display 206 provided in the sensor device 2, but also a display contained in or connected to a device which can acquire at least one of the detection result and the recognition result. A case where the behavior/state to be recognized is a heatstroke state will be herein described as an example.

FIG. 8A illustrates an example of a screen image 91A displayed when the candidate 83 for the heatstroke state is not detected. When the candidate 83 for the heatstroke state is not detected, the heat stress index 82 is less than the threshold value. Thus, the screen image 91A shows that the heat stress index 82 is “low”. In addition, the data subset 84 is not generated and not transmitted to the external processing device 3, and thus, the magnitude of the risk of heatstroke included in a recognition result by the external processing device 3 is not shown in the screen image 91A.

In a case where the screen image 91A is displayed, the data subset 84 is not generated and the wireless communication device 205 used to transmit the data subset 84 is not operating. It is therefore considered that the sensor device 2 is operating with low power consumption.

In contrast, FIG. 8B illustrates an example of a screen image 91B shown when the candidate 83 for the heatstroke state is detected. When the candidate 83 for the heatstroke state is detected, the heat stress index 82 is greater than or equal to the threshold value. Thus, the screen image 91B shows that the heat stress index 82 is “high”. In addition, in response to the detection of the candidate 83 for the heatstroke state, the data subset 84 is generated and transmitted to the external processing device 3, and then, a recognition result is received from the external processing device 3. Thus, the magnitude of the risk of heatstroke (in this case, 82%) included in the recognition result is shown in the screen image 91B.

A display mode of the characters representing the heat stress index 82 and the characters representing the magnitude of the risk of heatstroke may be changed. For example, the colors of these characters and the colors of the backgrounds of these characters may be changed in order that the greater the heat stress index 82 or the magnitude of the risk of heatstroke is, the more the user's attention can be attracted.

In a case where the screen image 91B is displayed, the power consumption for transmitting the data subset 84 to the external processing device 3 is necessary, whereas the highly accurate recognition of the heatstroke state (for example, estimation of the risk of heatstroke) can be performed in the external processing device 3. The highly accurate recognition may have great computational amount and require greater power consumption. Thus, a highly accurate recognition result on the behavior/state of the user wearing the sensor device 2 can be acquired without providing the sensor device 2 with a high-performance component that consumes great power, for performing a process having great computational amount.

Moreover, in the sensor device 2, the data subset 84 is generated and transmitted to the external processing device 3, only when a behavior/state candidate is detected. Accordingly, the power consumption can be reduced as compared to that in a case where the user's behavior/state is recognized with high accuracy or in a case where all the time-series sensor data 81 is transmitted to the external processing device 3. Thus, the sensor device 2 can be driven for a long time.

The sensor device 2 may detect candidates for, not only the above-described heatstroke state of the user, but also various behaviors/states such an unsteady posture and an unexpected action, and similarly, the external processing device 3 also may recognize various behaviors/states.

For example, the sensor device 2 detects a candidate for an unsteady posture of an operator (user), and transmits a data subset of time-series sensor data on triaxial acceleration to the external processing device 3. The external processing device 3 recognizes the operator's unsteady posture, and thereby estimates, for example, the risk of a fall. With this recognition result, the operator's attention can be attracted in accordance with, for example, the magnitude of the risk of the fall.

Moreover, for example, the sensor device 2 detects a candidate for the user's unexpected action that is not stated in an operations manual, and transmits a data subset of time-series sensor data on hexaxial acceleration and angular velocity to the external processing device 3. The external processing device 3 recognizes the operator's unexpected action, and thereby analyzes, for example, the action in detail. With this recognition result, for example, a warning not to perform an action not stated in the manual can be issued to the operator.

As described above, a candidate for the behavior/state to be recognized is detected by performing a simple process in the sensor device 2, and only when the candidate is detected, a subset of time-series sensor data is transmitted to the external processing device 3 via the wireless communication device 205 and a high-level recognition process is executed. The capability of recognizing a behavior/state thereby can be improved, using the external processing device 3 as necessary, while reducing the power consumption required for wireless communication.

### Second Embodiment

In the first embodiment, when a behavior/state candidate is detected, a data subset is generated and transmitted to the external processing device 3. In contrast, in a second embodiment, when a behavior/state candidate is detected and its degree of urgency is high, a data subset is generated and transmitted to the external processing device 3.

The configurations of a sensor device 2 and an external processing device 3 according to the second embodiment are the same as those of the sensor device 2 and the external processing device 3 of the first embodiment, respectively. The second embodiment differs from the first embodiment in that a degree-of-urgency calculation module for calculating the degree of urgency is added and the functions of the data subset generation module 30, the data subset transmission module 40, and the first behavior/state recognition module 50 are changed accordingly. In the following description, points differing from the first embodiment will be mainly explained.

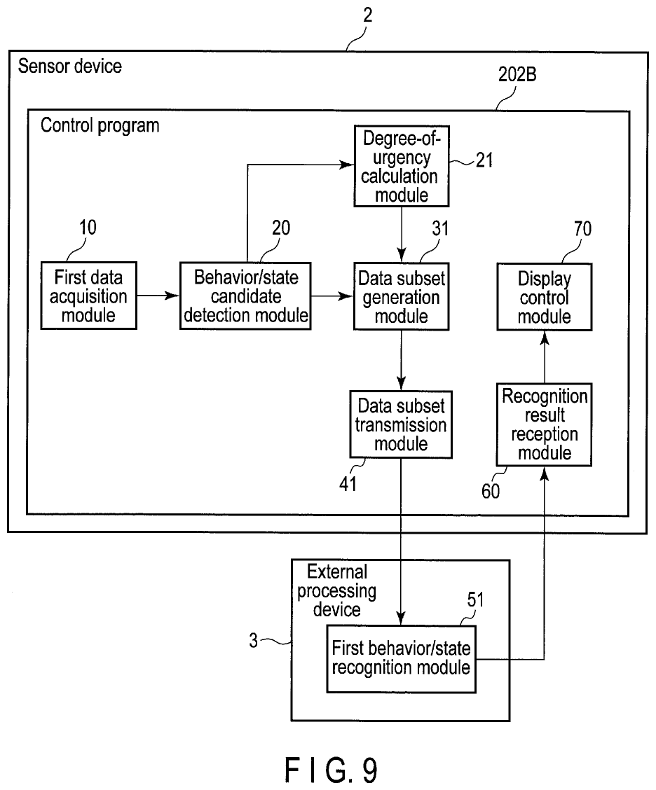

FIG. 9 illustrates a functional configuration of the sensor device 2 according to the second embodiment and the external processing device 3. As described above, the sensor device 2 further includes a degree-of-urgency calculation module 21, and includes a data subset generation module 31 and a data subset transmission module 41, which are configured by changing part of the functions of the data subset generation module 30 and the data subset transmission module 40 of the first embodiment, respectively. A first data acquisition module 10, a behavior/state candidate detection module 20, a recognition result reception module 60, and a display control module 70 operate as described in the first embodiment.

When a behavior/state candidate is detected, the degree-of-urgency calculation module 21 calculates the magnitude of its degree as the degree of urgency (or the degree of seriousness). For example, when a state in which the risk of a fall is high is detected as a behavior/state candidate, a variance value of an inclination of the user's waist or a level determined according to the magnitude of the variance value is used as the degree of urgency. In addition, for example, when a state in which the risk of heatstroke is high (heatstroke state) is detected as a behavior/state candidate, a heat stress index or a level determined according to the heat stress index is used as the degree of urgency.

When the degree of urgency indicates that a data subset should be generated and transmitted (for example, when the degree of urgency is greater than or equal to a threshold value), the data subset generation module 31 and the data subset transmission module 41 transmit a data subset of a specific period of at least one piece of one or more pieces of time-series sensor data to the external processing device 3, in accordance with the behavior/state candidate and the degree of urgency.

More specifically, when the behavior/state candidate detection module 20 has detected a behavior/state candidate, the data subset generation module 31 determines whether a data subset should be generated, in accordance with the degree of urgency calculated by the degree-of-urgency calculation module 21. When it is determined that a data subset should be generated, the data subset generation module 31 extracts a specific section from time-series sensor data, on the basis of the type of behavior/state candidate, the time (for example, time and date) when the behavior/state candidate is detected, and the degree of urgency, and thereby generates a data subset. The data subset generation module 31 selects, for example, at least one piece of time-series sensor data from one or more pieces of time-series sensor data, on the basis of the type of behavior/state candidate. The data subset generation module 31 then acquires data of a specific period based on the time when the behavior/state candidate is detected, of the selected at least one piece of time-series sensor data, as the data subset. The specific period is a period having a specific length, and including, for example, the time when the behavior/state candidate is detected. In the selection of the time-series sensor data and the determination of the specific period, during which data is should be extracted, the degree of urgency may be further taken into consideration.

The data subset transmission module 41 transmits the data subset generated by the data subset generation module 31 and the degree of urgency calculated by the degree-of-urgency calculation module 21 to the external processing device 3 via a wireless communication device 205.

In addition, as described above, the external processing device 3 of the second embodiment includes a first behavior/state recognition module 51, which is configured by changing part of the function of the first behavior/state recognition module 50 of the first embodiment.

The first behavior/state recognition module 51 receives a data subset and the degree of urgency from the sensor device 2. The first behavior/state recognition module 51 recognizes a user's behavior/state to be recognized, using the data subset and the degree of urgency. The first behavior/state recognition module 51 may store the recognition result in the external processing device 3 or transmit it to the sensor device 2, etc.

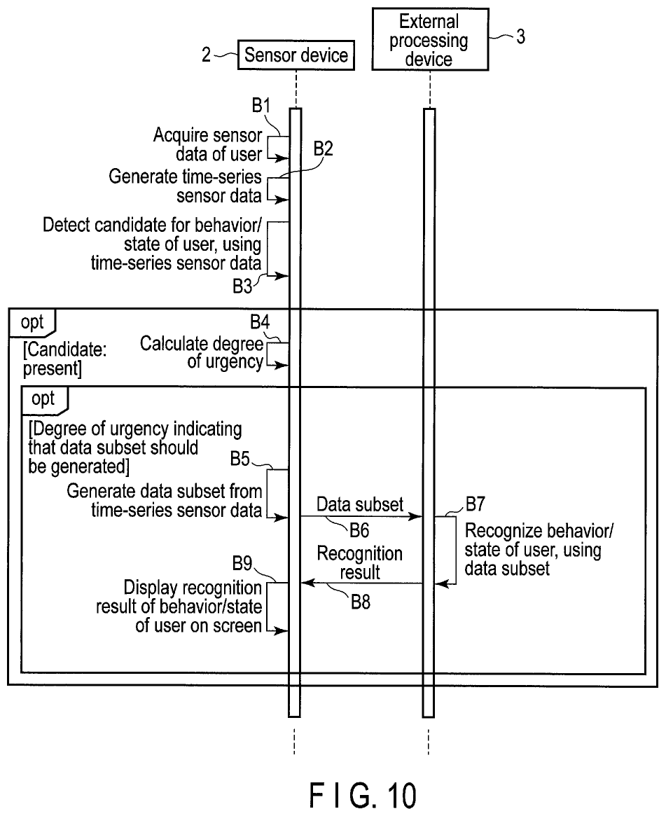

Next, an example of a processing sequence performed by the sensor device 2 and the external processing device 3 will be described with reference to FIG. 10. Steps B1, B2, and B3 illustrated in FIG. 10 are the same as steps A1, A2, and A3 described above with reference to FIG. 4.

When a behavior/state candidate has been detected in step B3, the degree-of-urgency calculation module 21 calculates the magnitude of the degree of the detected behavior/state candidate as the degree of urgency (B4).

When the calculated degree of urgency indicates that a data subset should be generated, the data subset generation module 31 extracts a specific section of time-series sensor data, on the basis of the type of behavior/state candidate, the time when the behavior/state candidate is detected, and the degree of urgency, and thereby generates a data subset (B5). In this case, the type and the number of channels of time-series sensor data from which the data subset is extracted, and the length and the position of the extracted data subset may vary according to the behavior/state candidate and the degree of urgency.

More specifically, the data subset generation module 31 determines whether a data subset should be generated, in accordance with the degree of urgency. The data subset generation module 31 determines whether a data subset should be generated, for example, in accordance with whether the degree of urgency is greater than or equal to a threshold value that is set for each type of detected behavior/state candidate. That is, in a case where a behavior/state candidate is detected, when the degree of urgency is greater than or equal to a threshold value associated with the behavior/state candidate, the data subset generation module 31 determines that a data subset should be generated. In contrast, when the degree of urgency is less than the threshold value, the data subset generation module 31 determines that a data subset should not be generated. When the data subset generation module 31 determines that a data subset should not be generated, the processing ends.

In addition, when the degree of urgency calculated by the degree-of-urgency calculation module 21 indicates that a data subset should be generated, and a data subset is generated by the data subset generation module 31, the data subset transmission module 41 transmits the data subset and the degree of urgency to the external processing device 3 via the wireless communication device 205 (B6).

The first behavior/state recognition module 51 of the external processing device 3 receives the data subset and the degree of urgency from the sensor device 2, and recognizes the user's behavior/state to be recognized, using the data subset and the degree of urgency (B7). In this case, an algorithm used to recognize the behavior/state may vary according to the degree of urgency. For example, when the degree of urgency is high, an algorithm for a detailed analysis is used, and when the degree of urgency is low, an algorithm for a simple analysis is used.

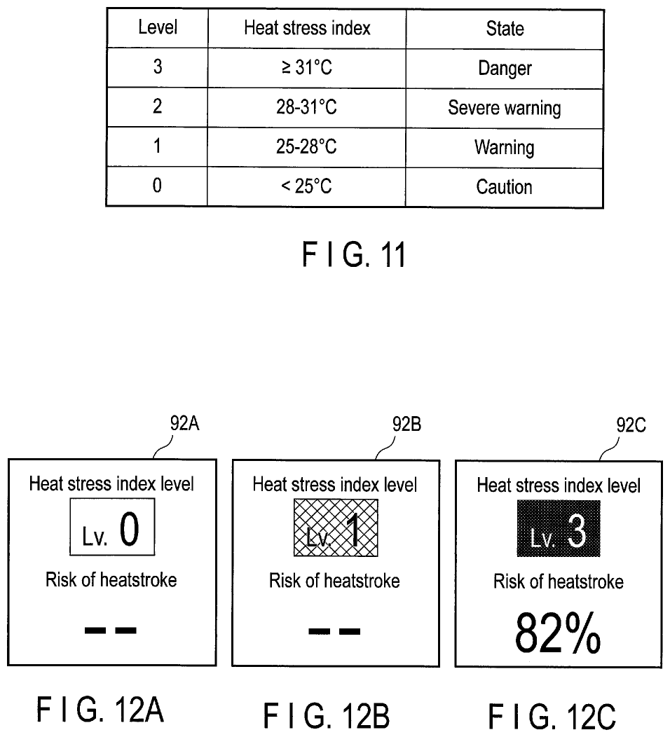

The operation in a case where the behavior/state to be recognized is a heatstroke state as in the case of the above-described example will be herein described with reference to FIG. 11, FIG. 12A, FIG. 12B, and FIG. 12C.

FIG. 11 illustrates an example of levels of heat stress indices used as the degrees of urgency. A heat stress index is classified into any one of levels according to its magnitude. In general, a warning on the heat is issued, on the basis of the level into which the heat stress index is classified. Thus, the level of the heat stress index may be used as the degree of urgency.

As illustrated in FIG. 11, for example, a heat stress index less than 25° C. is associated with level 0 (caution), a heat stress index greater than or equal to 25° C. but less than 28° C. is associated with level 1 (warning), a heat stress index greater than or equal to 28° C. but less than 31° C. is associated with level 2 (severe warning), and a heat stress index greater than or equal to 31° C. is associated with level 3 (danger).

The degree-of-urgency calculation module 21 calculates the degree of urgency corresponding to a heat stress index 82 calculated by the behavior/state candidate detection module 20, using information (for example, a table) indicating the relationship between heat stress indices and levels. The behavior/state candidate detection module 20 may detect, for example, the heat stress index 82 corresponding to level 1 or higher, as a behavior/state candidate (that is, a candidate 83 for the heatstroke state).

Then, the data subset generation module 31 determines whether the level of the heat stress index calculated as the degree of urgency is greater than or equal to a threshold value that is associated with the candidate for the heatstroke state (for example, level 2). When the level of the heat stress index is less than the threshold value, the data subset generation module 31 does not generate a data subset.

In contrast, when the level of the heat stress index is greater than or equal to the threshold value, the data subset generation module 31 generates a data subset, and the data subset transmission module 41 transmits the generated data subset to the external processing device 3. The first behavior/state recognition module 51 of the external processing device 3 recognizes the user's behavior/state, using the data subset, and transmits a recognition result to the sensor device 2, etc.

FIG. 12A, FIG. 12B, and FIG. 12C illustrate an example of screen images showing a detection result of a behavior/state candidate and a recognition result of a behavior/state with regard to the heatstroke state. The screen images illustrated in FIG. 12A, FIG. 12B, and FIG. 12C may be displayed on, not only a display 206 provided in the sensor device 2, but also a display contained in or connected to a device that can acquire at least one of a detection result and a recognition result. A case where the behavior/state to be recognized is a heatstroke state will be herein described as an example.

FIG. 12A illustrates an example of a screen image 92A displayed when the calculated heat stress index 82 corresponds to level 0. The screen image 92A shows that the heat stress index 82 is level 0. In addition, because the heat stress index 82 corresponding to level 0 is not detected as the candidate 83 for the heatstroke state, a data subset 84 is not generated and not transmitted to the external processing device 3. Thus, the magnitude of the risk of heatstroke, which is included in a recognition result obtained by the external processing device 3, is not shown in the screen image 92A.

FIG. 12B illustrates an example of a screen image 92B displayed when the calculated heat stress index 82 corresponds to level 1. The screen image 92B shows that the heat stress index 82 is level 1. The display mode of level 1 may be different from that of level 0, which is lower than level 1. For example, the user's attention can be attracted by changing the color of characters or the color of the background.

In addition, as in the case of FIG. 12A, the heat stress index 82 corresponding to level 1 is detected as the candidate 83 for the heatstroke state, but it is determined that its degree of urgency does not indicate that a data subset should be generated. Thus, the data subset 84 is not generated and not transmitted to the external processing device 3. Thus, the magnitude of the risk of heatstroke, which is included in a recognition result obtained by the external processing device 3, is not shown in the screen image 92B.

FIG. 12C illustrates an example of a screen image 92C displayed when the calculated heat stress index 82 corresponds to level 2 or higher. The screen image 92C shows that the heat stress index 82 is level 3. The heat stress index 82 corresponding to level 2 or higher is detected as the candidate 83 for the heatstroke state, and it is determined that its degree of urgency indicates that the data subset 84 should be generated. Then, after the data subset 84 is generated and transmitted to the external processing device 3, a recognition result is received from the external processing device 3. Thus, the magnitude of the risk of heatstroke (in this case, 82%), which is included in the recognition result, is shown in the screen image 92C.

The display mode of level 2 or higher of the heat stress indices may be different from those of level 1 and level 0. For example, the color of characters or the color of the background is changed (for example, the colors of the characters and the background are displayed in reverse), and the magnitude of the risk of heatstroke calculated by the external processing device 3 with high accuracy is shown, so that the user's maximum attention can be attracted.

The sensor device 2 may detect candidates for, not only the above-described heatstroke state of the user, but also various behaviors/states such an unsteady posture and an unexpected action, and similarly, the external processing device 3 also may recognize various behaviors/states.

For example, the sensor device 2 detects a candidate for an unsteady posture of an operator (user), and only when its degree of urgency is high, transmits a data subset of time-series sensor data on triaxial acceleration to the external processing device 3. The external processing device 3 recognizes the operator's unsteady posture, and thereby estimates, for example, the risk of a fall. With this recognition result, the operator's attention can be attracted in accordance with, for example, the magnitude of the risk of the fall.

Moreover, for example, the sensor device 2 detects a candidate for an unexpected action of the user which is not stated in an operations manual, and only when its degree of urgency is high, transmits a data subset of time-series sensor data on hexaxial acceleration and angular velocity to the external processing device 3. The external processing device 3 recognizes the operator's unexpected action, and thereby analyzes, for example, the action in detail. With this recognition result, for example, a warning can be issued to the operator.

As described above, in the second embodiment, the processing can be changed in accordance with the degree of urgency by further adding a component for calculating the degree of urgency of a candidate for the behavior/state to be recognized. The generation of a data subset and the transmission of a data subset via the wireless communication device 205 thereby can be performed only when the degree of urgency is high. Thus, the power consumption for wireless communication can be further reduced. Accordingly, the sensor device 2 can be driven for a longer time, and a highly accurate recognition result of a behavior/state can be acquired.

### Third Embodiment

In the first embodiment, when a behavior/state candidate is detected, a data subset is generated and transmitted to the external processing device 3. Moreover, in the second embodiment, when a behavior/state candidate is detected and its degree of urgency is high, a data subset is generated and transmitted to the external processing device 3. In contrast, in a third embodiment, whether a data subset can be transmitted via the wireless communication device 205 is further taken into consideration.

The configurations of a sensor device 2 and an external processing device 3 according to the third embodiment are the same as those of the sensor devices 2 and the external processing devices 3 of the first and second embodiments, respectively. The third embodiment differs from the first and second embodiments in that a second behavior/state recognition module 52 is added and the function of the data subset transmission module 41 is changed accordingly. In the following description, points differing from the first and second embodiments will be mainly explained.

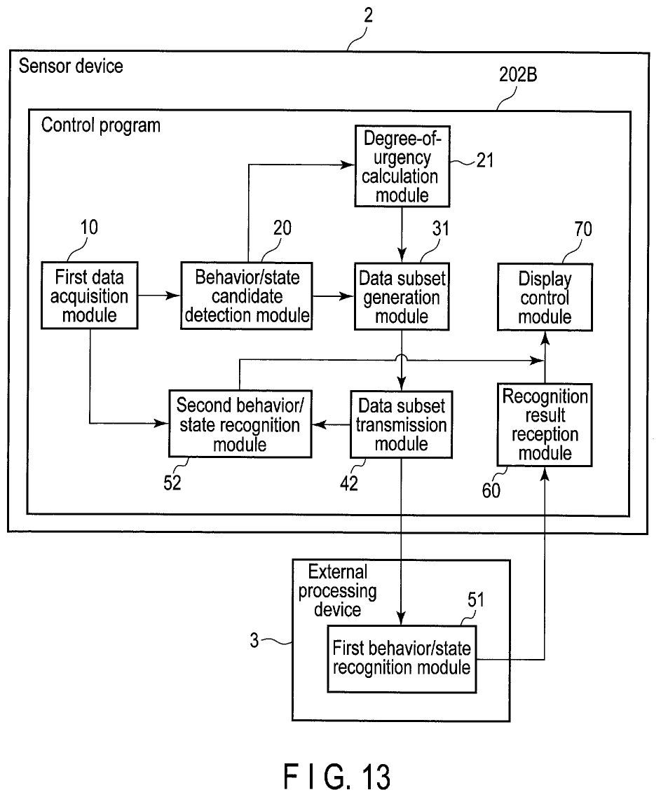

FIG. 13 illustrates a functional configuration of the sensor device 2 and the external processing device 3 according to the third embodiment. As described above, the sensor device 2 further includes the second behavior/state recognition module 52, and includes a data subset transmission module 42, configured by changing part of the function of the data subset transmission module 41 of the second embodiment. A first data acquisition module 10, a behavior/state candidate detection module 20, a degree-of-urgency calculation module 21, a data subset generation module 31, a recognition result reception module 60, and a display control module 70 operate as described in the first and second embodiments. In addition, a first behavior/state recognition module 51 in the external processing device 3 also operate as described in the first and second embodiments.

The data subset transmission module 42 attempts to transmit a data subset generated by the data subset generation module 31 and the degree of urgency calculated by the degree-of-urgency calculation module 21. At that time, it is confirmed whether communications (or connection) via a wireless communication device 205 are available. When communications via the wireless communication device 205 are available, the data subset transmission module 42 transmits the data subset and the degree of urgency to the external processing device 3 via the wireless communication device 205.

When communications via the wireless communication device 205 are unavailable, the data subset transmission module 42 sends the data subset to the second behavior/state recognition module 52.

When the data subset cannot be transmitted to the external processing device 3, the second behavior/state recognition module 52 recognizes a behavior/state of a user, using the data subset or using one or more pieces of time-series sensor data generated by the first data acquisition module 10.

More specifically, the second behavior/state recognition module 52 executes an alternative process for recognizing the user's behavior/state, using the data subset or using the one or more pieces of time-series sensor data. This alternative process replaces the process executed by the first behavior/state recognition module 51, and is, for example, a process involving a lower computational cost than that of the process executed by the first behavior/state recognition module 51. However, the alternative process is not limited to this example. In the alternative process, for example, the user's behavior and state are recognized in more detail than in the process executed by the behavior/state candidate detection module 20, whereas its recognition accuracy may be lower than that of the process executed by the first behavior/state recognition module 51.

When communications via the wireless communication device 205 are unavailable, the data subset transmission module 42 may send the data subset and the degree of urgency to the second behavior/state recognition module 52. The second behavior/state recognition module 52 may recognize the user's behavior/state, using the data subset and the degree of urgency.

Alternatively, when communications via the wireless communication device 205 are unavailable, the second behavior/state recognition module 52 may recognize the user's behavior/state, using data of a certain section of time-series sensor data generated by the first data acquisition module 10 and the behavior/state candidate detection module 20. At that time, the second behavior/state recognition module 52 may use at least one of a behavior/state candidate detected by the behavior/state candidate detection module 20 and the degree of urgency calculated by the degree-of-urgency calculation module 21.

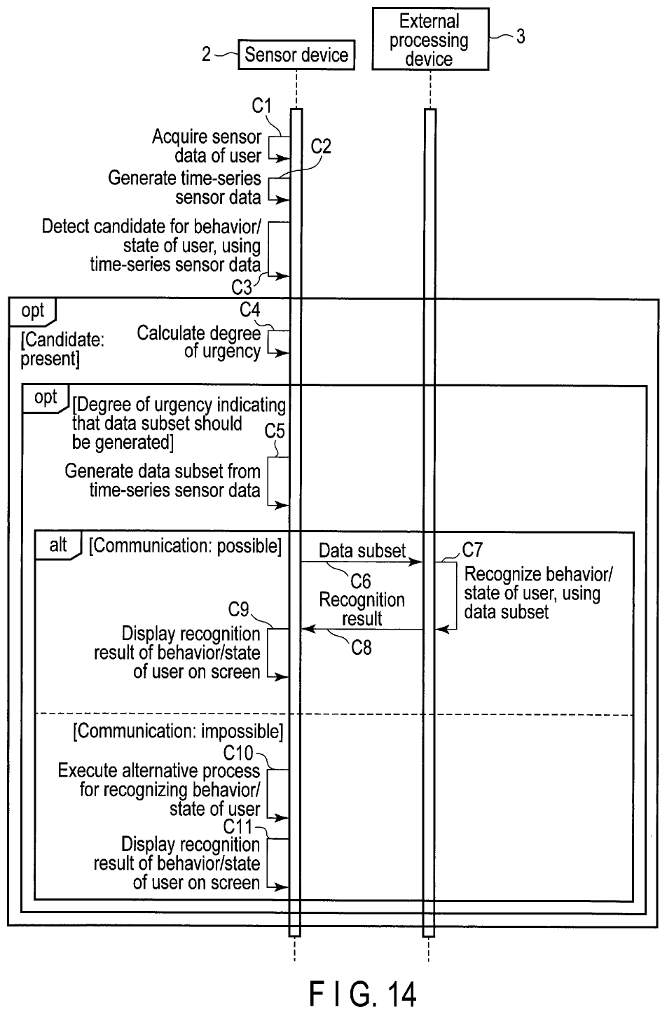

Next, an example of a processing sequence performed by the sensor device 2 and the external processing device 3 will be described with reference to FIG. 14. Steps C1, C2, and C3 shown in FIG. 14 are the same as steps A1, A2, and A3 described above with reference to FIG. 4, respectively. In addition, steps C4 and C5 shown in FIC. 14 are the same as steps B4 and B5 described above with reference to FIG. 10, respectively.

When a data subset has been generated in step C5, the data subset transmission module 42 determines whether wireless communication via the wireless communication device 205 is possible, and when it is possible, transmits the data subset and the degree of urgency to the external processing device 3 (C6). Subsequent steps C7, C8, and C9 are the same as steps B7, B8, and B9 described above with reference to FIG. 10, respectively.

In contrast, when a data subset has been generated in step C5 and wireless communication via the wireless communication device 205 is impossible, the second behavior/state recognition module 52 executes the alternative process for simply recognizing the user's behavior/state (C10). Then, the display control module 70 displays a recognition result of the alternative process on a screen of a display 206 (C11). That is, the recognition result obtained by the second behavior/state recognition module 52 is displayed instead of a recognition result obtained by the external processing device 3.

In the first and second embodiments, when a data subset cannot be transmitted to the external processing device 3 via the wireless communication device 205, the external processing device 3 cannot recognize the user's behavior/state, using the data subset. Thus, a recognition result cannot be displayed in the sensor device 2, etc.

In contrast, in the third embodiment, even when wireless communication via the wireless communication device 205 is impossible, a simple recognition process (alternative process) is executed by the second behavior/state recognition module 52 in the sensor device 2, and a recognition result can be displayed.

For example, when the behavior/state to be recognized is a heatstroke state, the second behavior/state recognition module 52 executes the alternative process of acquiring the risk of heatstroke corresponding to a heat stress index calculated by the behavior/state candidate detection module 20, using a table. The table includes records each including a heat stress index and the risk of heatstroke associated with the heat stress index. This table is prepared in advance, and indicates the relationship between heat stress indices and the risks of heatstroke. The relationship is statistically calculated in advance. The second behavior/state recognition module 52 can obtain a simple recognition result merely by acquiring the risk of heatstroke corresponding to a heat stress index from the table.

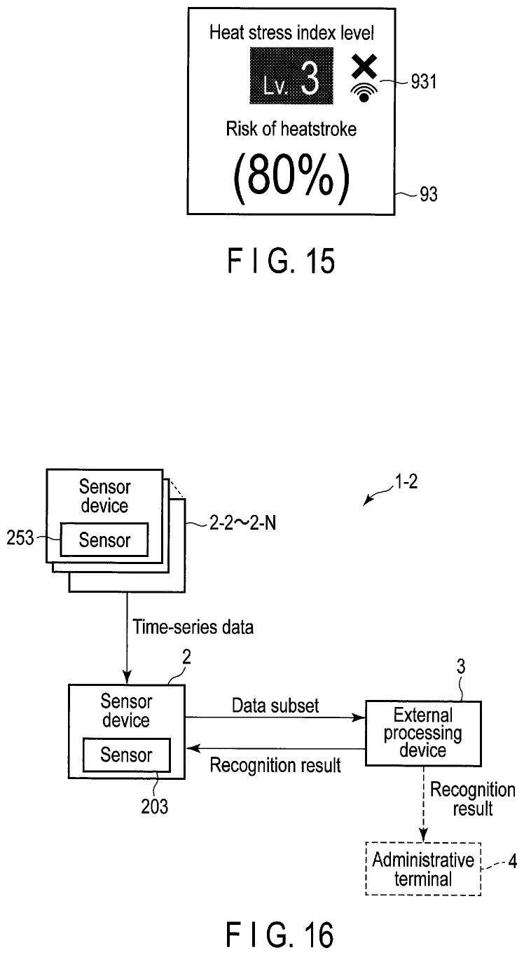

FIG. 15 illustrates an example of a screen image 93 showing a recognition result obtained by the second behavior/state recognition module 52. A case where the behavior/state to be recognized is a heatstroke state is herein described as an example.

The screen image 93 shows that a heat stress index 82 is level 3. The heat stress index 82 corresponding to level 2 or higher is detected as a candidate 83 for the heatstroke state, and it is determined that its degree of urgency indicates that a data subset should be generated. Then, because a data subset 84 is generated but communication via the wireless communication device 205 is impossible, the magnitude of the risk of heatstroke (in this case, 80%) based on a recognition result, which is obtained by the alternative process executed by the second behavior/state recognition module 52, is displayed in the screen image 93.

The risk of heatstroke calculated by the alternative process may be inferior in accuracy to the risk of heatstroke calculated by the external processing device 3. Thus, the risk of heatstroke may be displayed in a mode varying according to its accuracy in order that the user can distinguish whether the displayed risk of heatstroke is calculated by the alternative process or by the external processing device 3, that is, in order that the user can judge the accuracy of the risk of heatstroke. For example, an icon 931 indicating that wireless communications are unavailable may be displayed in the screen image 93. Moreover, for example, the risk of heatstroke calculated by the alternative process is displayed in parentheses as shown in the screen image 93. In this manner, the risk of heatstroke calculated by the alternative process may be displayed to show that it is a reference value, not a reliable value.

As described above, in the third embodiment, a component for simply recognizing a behavior/state is further added. Thus, when the wireless communication device 205 is unavailable, for example, when the quality of wireless communication temporarily deteriorates in a field workplace where the signal is bad, etc., a behavior/state can be continuously recognized by executing the simple and light alternative process, and its recognition result can be presented to the user. Accordingly, while the reliability of a recognition result may temporarily decline, a behavior/state can be recognized at all times.

The data subset transmission module 42 and the second behavior/state recognition module 52 described above can be similarly applied also to the sensor device 2 of the first embodiment (that is, the sensor device 2 that does not include the degree-of-urgency calculation module 21).

### Fourth Embodiment

In the first embodiment, when a behavior/state candidate is detected, a data subset is generated and transmitted to the external processing device 3. In the second embodiment, when a behavior/state candidate is detected and its degree of urgency is high, a data subset is generated and transmitted to the external processing device 3. Moreover, in the third embodiment, whether a data subset can be transmitted via the wireless communication device 205 is further taken into consideration. In contrast, in a fourth embodiment, sensor data may be acquired also from a sensor device other than the sensor device 2.

The configurations of a sensor device 2 and an external processing device 3 according to the fourth embodiment are the same as those of the sensor devices 2 and the external processing devices 3 of the first to third embodiments, respectively. The fourth embodiment differs from the first to third embodiments in that the function of the data subset generation module 31 is changed. In the following description, points differing from the first to third embodiments will be mainly explained.

FIG. 16 illustrates a configuration of an information processing system 1-2 including the sensor device 2 according to the fourth embodiment. The information processing system 1-2 further includes one or more sensor devices 2-2 to 2-N other than the sensor device 2, as compared to the information processing systems 1 of the first to third embodiments.

A user wears the one or more sensor devices 2-2 to 2-N on, for example, a region differing from that of the sensor device 2. Each of the sensor devices 2-2 to 2-N acquires, for example, sensor data related to the region on which it is worn, by a sensor 253 contained therein. The acquired sensor data may include various types of data effective in recognizing the user's behavior/state as in the case of sensor data acquired by the sensor device 2. Each of the sensor devices 2-2 to 2-N has, for example, the same system configuration as that of the sensor device 2. Each of the sensor devices 2-2 to 2-N is not necessarily a sensor device worn by the user, but may be any sensor device that can observe the user, for example, a sensor device installed in a place (for example, a battery-driven video camera).

In the following description, for the sake of clarification, the sensor device 2 according to the fourth embodiment will be referred to as a first sensor device 2, and a sensor device of the one or more sensor devices 2-2 to 2-N will be referred to also as an i-th sensor device 2-i. In this case, i and N are integers greater than or equal to two.

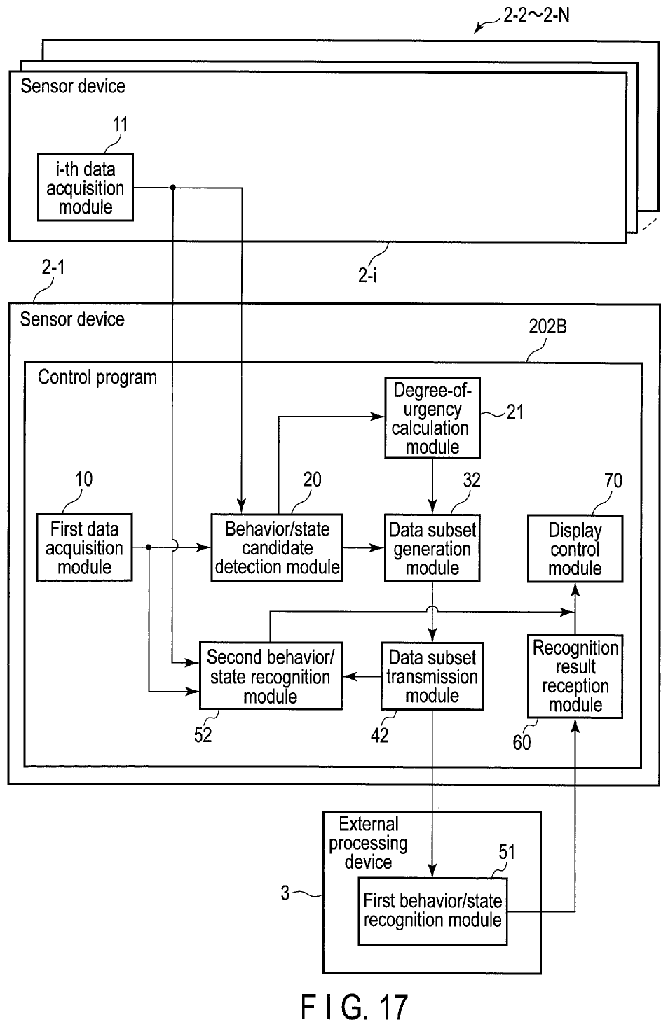

FIG. 17 illustrates a functional configuration of the first sensor device 2, the external processing device 3, and the i-th sensor device 2-i. The one or more sensor devices 2-2 to 2-N may have the same functional configuration, and may operate in the same way.

As described above, the first sensor device 2 includes a data subset generation module 32, which is configured by changing part of the function of the data subset generation module 31 of the second embodiment. A first data acquisition module 10, a behavior/state candidate detection module 20, a degree-of-urgency calculation module 21, a data subset transmission module 42, a recognition result reception module 60, and a display control module 70 operate as described in the first to third embodiments. In addition, a first behavior/state recognition module 51 in the external processing device 3 operates as described in the first to third embodiments.

The i-th sensor device 2-i includes an i-th data acquisition module 11. The i-th data acquisition module 11 has the same function as that of the first data acquisition module 10 in the first sensor device 2. That is, the i-th data acquisition module 11 acquires sensor data with the sensor 253 in the i-th sensor device 2-i, and generates time-series sensor data into which pieces of sensor data, which are acquired at points in time, are combined. The i-th data acquisition module 11 may transmit the generated time-series sensor data to the first sensor device 2. The i-th data acquisition module 11 may transmit the time-series sensor data to the first sensor device 2, for example, using wireless communication of a wireless LAN or Bluetooth.

When a behavior/state candidate has been detected, the data subset generation module 32 and the data subset transmission module 42 may acquire one or more pieces of time-series sensor data from the i-th data acquisition module 11 in the i-th sensor device 2-i and transmit a data subset of a specific period of at least one of the one or more pieces of time-series sensor data to the external processing device 3, in accordance with the behavior/state candidate, or in accordance with the behavior/state candidate and its degree of urgency.

More specifically, when the behavior/state candidate detection module 20 has detected a behavior/state candidate, the data subset generation module 32 determines whether a data subset should be generated, on the basis of the degree of urgency calculated by the degree-of-urgency calculation module 21. When it is determined that a data subset should be generated, the data subset generation module 31 extracts a specific section from time-series sensor data, on the basis of the type of behavior/state candidate, the time (for example, time and date) when the behavior/state candidate is detected, and the degree of urgency, and thereby generates a data subset.

The data subset generation module 32 selects at least one piece of time-series sensor data from multiple pieces of time-series sensor data, in accordance with, for example, the type of behavior/state candidate. The multiple pieces of time-series sensor data include not only time-series sensor data that the data acquisition module 10 generates using sensor data, but also time-series sensor data that the data acquisition modules 11 in the other one or more sensor devices 2-2 to 2-N generate using sensor data. The data subset generation module 32 acquires data of a specific period based on the time when the behavior/state candidate is detected, of the selected at least one piece of time-series sensor data, as the data subset. The specific period has a specific length, and includes, for example, the time when the behavior/state candidate is detected. In the selection of the time-series sensor data and the determination of the specific period, during which data is should be extracted, the degree of urgency may be further taken into consideration.

The behavior/state candidate detection module 20 and the second behavior/state recognition module 52 of the first sensor device 2 may execute the respective processes, using time-series sensor data transmitted by the i-th data acquisition module 11, in addition to time-series sensor data output from the first data acquisition module 10 or instead of the time-series sensor data output from the first data acquisition module 10.

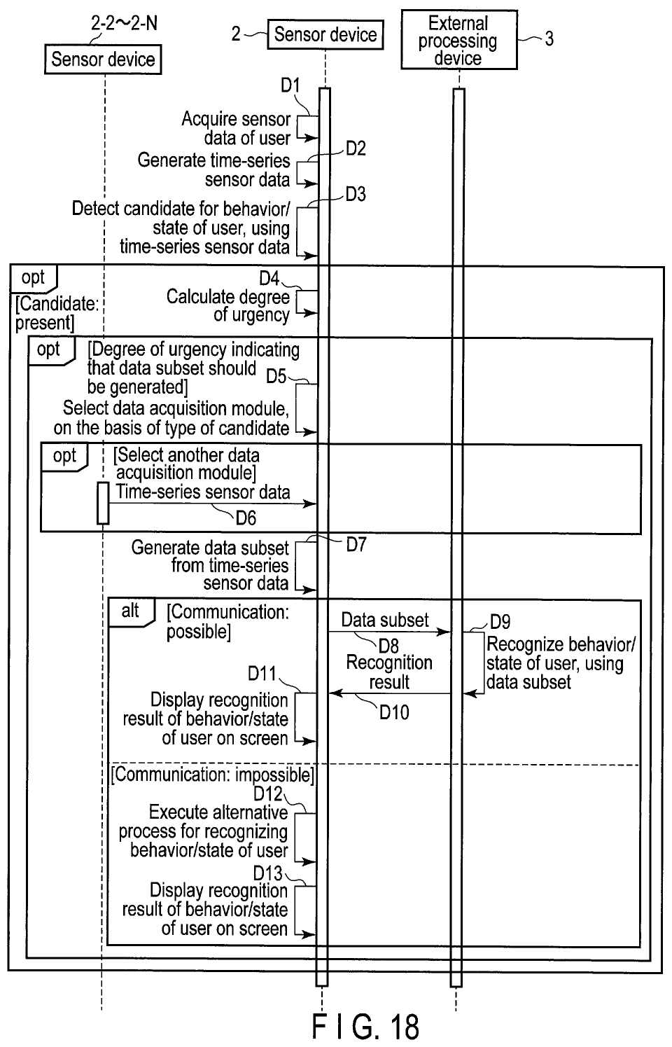

Next, an example of a processing sequence performed by the first sensor device 2, the external processing device 3, and the other one or more sensor devices 2-2 to 2-N will be described with reference to FIG. 18. Steps D1, D2, and D3 shown in FIG. 18 are the same as steps A1, A2, and A3 described above with reference to FIG. 4. In addition, step D4 shown in FIG. 18 is the same as step B4 described above with reference to FIG. 10.

When the degree of urgency indicating that a data subset should be generated has been calculated in step D4, the data subset generation module 32 selects a data acquisition module used to generate a data subset from the first data acquisition module 10 in the first sensor device 2 and the i-th data acquisition module 11 included in each of the one or more sensor devices 2-2 to 2-N, on the basis of the behavior/state candidate and its degree of urgency (D5). The data subset generation module 32 may select multiple data acquisition modules.

When a data acquisition module other than the first data acquisition module 10 in the first sensor device 2 is selected, that is, when the i-th data acquisition module 11 included in any one of the one or more sensor devices 2-2 to 2-N is selected, the data subset generation module 32 receives time-series sensor data from the selected i-th data acquisition module 11 (D6).

For example, it is assumed that the first sensor device 2 is worn on the user's wrist and a candidate for a behavior/state is detected from first time-series sensor data obtained by the first data acquisition module 10 in the first sensor device 2. This candidate for the behavior/state can be detected from the first time-series sensor data. However, in order Lo recognize the behavior/state accurately, not time-series sensor data of the wrist but time-series sensor data of the waist, for example, is necessary. In this case, the data subset generation module 32 selects the i-th data acquisition module 11 in the i-th sensor device 2-i worn on the user's waist, and receives i-th time-series sensor data from the i-th data acquisition module 11.

The data subset generation module 32 generates a data subset, using time-series sensor data acquired from at least any one of the first data acquisition module 10 and the one or more i-th data acquisition modules 11 included in the one or more sensor devices 2-2 to 2-N, respectively (D7). The data subset generation module 32 extracts a specific section of the time-series sensor data, on the basis of the type of behavior/state candidate, the time when the behavior/state candidate is detected, and the degree of urgency, and thereby generates the data subset. In this case, the type and the number of channels of time-series sensor data from which the data subset is extracted, and the length and the position of the extracted data subset may vary according to the behavior/state candidate and the degree of urgency. Subsequent steps D8 to D13 are the same as steps C6 to C11 described above with reference to FIG. 14.

As described above, in the fourth embodiment, time-series sensor data acquired from the one or more sensor devices 2-2 to 2-N other than the first sensor device 2 is also additionally used, and in accordance with a detected candidate for a behavior/state, time-series sensor data suitable for the behavior/state can be selected and used for recognition. For example, when the user wears the sensor devices 2 and 2-2 to 2-N on multiple regions, respectively, time-series sensor data acquired by the sensor device on the region suitable for recognizing a behavior/state accurately is used for recognition. Thus, the accuracy in recognizing a behavior/state is improved, and power consumption can be reduced by acquiring time-series sensor data from the other sensor devices 2-2 to 2-N only when a behavior/state candidate is detected.

The above-described data subset generation module 32 may be similarly applied also to the sensor device 2 of the first embodiment (that is, the sensor device 2 that does not include the degree-of-urgency calculation module 21) and the sensor device 2 of the second embodiment (that is, the sensor device 2 that does not include the second behavior/state recognition module 52).

As described above, according to the first to fourth embodiments, long-time driving becomes possible and a highly accurate recognition result of a behavior/state can be acquired. The first data acquisition module 10 and the behavior/state candidate detection module 20 acquire one or more pieces of time-series sensor data, using the one or more sensors 203, and detects a behavior/state candidate of the user wearing or carrying the sensor device 2, using at least one of the one or more pieces of time-series sensor data. When the behavior/state candidate is detected, the data subset generation module 30 and the data subset transmission module 40 transmit a data subset of a first period, of the at least one of the one or more pieces of time-series sensor data, to the external processing device 3, in accordance with the behavior/state candidate.

In this manner, the sensor device 2 executes the process of detecting a behavior/state candidate, which has low computational amount, and only when it is determined that a high-level process is necessary, transmits a data subset to the external processing device 3. Thus, the amount of data transmitted to the external processing device 3 is reduced without compressing time-series sensor data, such as removing several frequency components, and the long-time driving of the sensor device and an improvement in the accuracy in detecting a behavior/state can be realized at the same time.

Each of the various functions disclosed in the first to fourth embodiments may be realized by a circuit (processing circuit). Examples of the processing circuit include a programmed processor such as a central processing unit (CPU). This processor performs each described function by executing a computer program (instructions) stored in a memory. This processor may be a microprocessor including an electronic circuit. Examples of the processing circuit also include a digital signal processor (DSP), an application-specific integrated circuit (ASIC), a microcontroller, a controller, and other electronic circuit components. Each of the components other than the CPU disclosed in the embodiments also may be realized by the processing circuit.

Since various processes of the first to fourth embodiments can be realized by a computer program, the same advantages as those of the embodiments can easily be obtained simply by installing the computer program in a computer through a computer-readable storage medium in which the computer program is stored and by executing the computer program.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An electronic apparatus that is wearable or portable by a user, comprising:
one or more sensors;
one or more processors configured to:
acquire one or more pieces of first time-series sensor data, using the one or more sensors; and
detect a candidate for at least one of a behavior or a state of the user, using at least one of the one or more pieces of first time-series sensor data; and
a transmitter configured to transmit, when the candidate is detected, a data subset of a first period, of at least one of the one or more pieces of first time-series sensor data, to an external processing device, in accordance with the candidate.

2. The electronic apparatus of claim 1, further comprising
a receiver configured to receive a recognition result of at least one of a behavior and a state of the user, which is recognized using the data subset, from the external processing device.

3. The electronic apparatus of claim 2, further comprising
a display controller configured to display the recognition result on a screen.

4. The electronic apparatus of claim 1, further comprising
a display controller configured to display information on the candidate on a screen.

5. The electronic apparatus of claim 1, wherein
the one or more processors are further configured to calculate a degree of urgency of the candidate, and
the transmitter is configured to transmit, when the degree of urgency is greater than or equal to a threshold value, the data subset of the first period, of at least one of the one or more pieces of first time-series sensor data, to the external processing device, in accordance with the candidate and the degree of urgency.

6. The electronic apparatus of claim 5, wherein
the transmitter is further configured to, when the candidate is detected,
acquire one or more pieces of second time-series sensor data from an another electronic apparatus other than the electronic apparatus, in accordance with the candidate and the degree of urgency, and
transmit a data subset of the first period, of at least one of the one or more pieces of second time-series sensor data, to the external processing device, in accordance with the candidate.

7. The electronic apparatus of claim 1, wherein
the transmitter is further configured to, when the candidate is detected,
acquire one or more pieces of second time-series sensor data from an another electronic apparatus other than the electronic apparatus, in accordance with the candidate, and
transmit a data subset of the first period, of at least one of the one or more pieces of second time-series sensor data, to the external processing device, in accordance with the candidate.

8. The electronic apparatus of claim 1, wherein
the one or more processors are further configured to recognize, when the data subset is not transmittable to the external processing device, at least one of a behavior and a state of the user, using the data subset or using the one or more pieces of first time-series sensor data.

9. The electronic apparatus of claim 8, further comprising
a display controller configured to display the at least one of a behavior and a state of the user recognized by the one or more processors on a screen.

10. An information processing system comprising an electronic apparatus that is wearable or portable by a user and an information processing device, wherein
the electronic apparatus comprises one or more sensors,
the electronic apparatus is configured to:
acquire one or more pieces of time-series sensor data, using the one or more sensors;
detect a candidate for at least one of a behavior and a state of the user, using at least one of the one or more pieces of time-series sensor data; and
transmit a data subset of a first period, of at least one of the one or more pieces of time-series sensor data, to the information processing device, in accordance with the candidate, and
the information processing device is configured to recognize at least one of a behavior and a state of the user, using the data subset.
